(19) United States
(12) Patent Application Publication
(10) Pub. No.: US 2006/0135896 A1
(43) Pub. Date: Jun. 22, 2006

(54) Electrode with integrated pull-tab

(75) Inventor: Russell A. Latimer, Sunbury, OH (US)

Correspondence Address: MCDERMOTT WILL & EMERY LLP, 18191 VON KARMAN AVE., SUITE 500, IRVINE, CA, 92612-7108, US

(73) Assignee: Vision Quest Industries, Inc., Irvine, CA (US)

(21) Appl. No.: 11/281,602

(22) Filed: Nov. 18, 2005

Related U.S. Application Data
(60) Provisional application No. 60/637,326, filed on Dec. 17, 2004.

(51) Int. Cl. A61F 5/00

(52) U.S. Cl. 602/2; 602/3

(57) ABSTRACT

An electrode for delivering a stimulation signal to a user, the electrode including a conductive layer having an adhesive surface on a first side and a conductive material on a second side, a laminate layer having an adhesive material on a first side and being disposed over the second side of the conductive layer such that the first side of the laminate layer adheres to the second side of the conductive layer, an input wire disposed between the conductive layer and the laminate layer, the input wire having a connection end and an external end, the connection end being disposed on the second side of the conductive layer and the external end being provided beyond the boundaries of the conductive layer and the laminate layer, and a pull-tab layer disposed between the conductive layer and the laminate layer, a disc portion with an adhesive surface being provided at a first end of the pull-tab layer, and a grip portion being provided at a second end of the pull-tab layer, the adhesive surface of the disc portion being disposed over the connection end of the input wire on the conductive layer, and the grip portion extending beyond the boundaries of the conductive layer and the laminate layer.

### CROSS-REFERENCES TO RELATED APPLICATIONS

This application claims priority to U.S. Provisional Patent Application No. 60/637,326, filed Dec. 17, 2004, titled “Electrode With Integrated Pull Tab,” which is incorporated by reference herein in its entirety.

### BACKGROUND OF THE INVENTION

1. Field of the Invention

The present invention concerns an electrode for delivering an electric stimulation for therapeutic purposes. In particular, the electrode incorporates an integrated pull-tab for structurally securing an input wire to the electrode and for use in removal of the electrode.

2. Description of the Related Art

Known types of electrodes are usually round and have a sticky surface on the bottom for applying the electrode to the skin of the user. Such electrodes also have an input wire attached in some fashion to the electrode, wherein the other end of the wire is connected to an electric power source for generating the stimulation signal to the user.

When a commonly known electrode is used, the user typically pulls a liner off of the bottom of the electrode to expose the sticky underside of the electrode for application to the skin. In addition, electrodes are often used more than once, which requires the user to remove the electrode from the skin and then re-apply a backing to the sticky surface of the electrode until the electrode is used next. Problems often arise with such electrodes when removing them from the skin because they are very adhesive and can be difficult to remove.

A user may try to remove the electrode by trying to pry up an edge of the electrode and then peeling the electrode up off of the skin. This removal process can often be frustrating and difficult for the user. In addition, the application and removal of the typical electrode from the skin can result in excessive stress on the connection point where the input wire is attached to the electrode, and may result in the wire becoming separated from the electrode and/or causing other damage to the electrode.

Although, others have attempted to solve these problems by various means, such solutions have been approached separately. In addition, existing attempts to provide a pull-tab result in excessive material and labor costs, while existing attempts to secure the wire connection to the electrode often require a complex electrode connection or excessive material.

Accordingly, it is desirable to develop an electrode that solves both of the foregoing problems with a single solution in an efficient and cost-effective manner.

### SUMMARY OF THE INVENTION

The present invention addresses the foregoing need by providing an electrode with an integrated pull-tab for structurally securing an input wire to the electrode and for use in easily removal of the electrode.

In particular, according to one aspect of the invention, the present invention is directed to an electrode for delivering a stimulation signal to a user, the electrode including a conductive layer having an adhesive surface on a first side and a conductive material on a second side, and a laminate layer having an adhesive material on a first side and being disposed over the second side of the conductive layer such that the first side of the laminate layer adheres to the second side of the conductive layer. The electrode also includes an input wire disposed between the conductive layer and the laminate layer, the input wire having a connection end and an external end, the connection end being disposed on the second side of the conductive layer and the external end being provided beyond the boundaries of the conductive layer and the laminate layer. A pull-tab layer is disposed between the conductive layer and the laminate layer, a disc portion with an adhesive surface being provided at a first end of the pull-tab layer, and a grip portion being provided at a second end of the pull-tab layer, the adhesive surface of the disc portion being disposed over the connection end of the input wire on the conductive layer, and the grip portion extending beyond the boundaries of the conductive layer and the laminate layer.

Preferably, the electrode also includes a removable liner layer which is in contact with the adhesive layer of the conductive layer to maintain the adhesive layer in a good condition until its next use. The removable liner layer can be made of a mylar or a polyester material. Also, the conductive layer and the laminate layer are disc shaped and are substantially the same size. The connection end of the input wire and the disc portion of the pull-tab layer are positioned over the center of the conductive surface of the conductive layer. The adhesive surface of the conductive layer is preferably a conductive hydrogel. The conductive layer is preferably comprised of a carbon film and the conductive surface includes a silver-based film.

In this manner, the present invention provides an electrode with an integrated pull-tab for structurally securing an input wire to the electrode and for use in easy removal of the electrode. The integrated pull-tab simultaneously solves the problems of maintaining a secure connection of the input wire to the electrode during repeated use, and of providing a grip tab for the user to grab for easy removal of the electrode from the removable liner or from the skin. Most importantly, the integrated pull-tab simultaneously solves the foregoing problems in an efficient and cost effective manner.

These and further objects and advantages of the present invention will be understood and appreciated by those skilled in the art by reference to the following specification, claims, and drawings.

### BRIEF DESCRIPTION OF THE DRAWINGS

FIG. 1 is a schematic diagram depicting an electrode with an integrated pull-tab according to one embodiment of the present invention.

FIG. 2 is a schematic diagram depicting the integrated pull-tab according to one embodiment of the present invention.

FIG. 3 is a schematic diagram depicting a cross section of the electrode with an integrated pull-tab according to one embodiment of the present invention.

FIGS. 4A to 4D are schematic diagrams for explaining the assembly of the electrode with an integrated pull-tab according to one embodiment of the present invention.

### DETAILED DESCRIPTION OF THE INVENTION

As mentioned above, the present invention is generally directed to an electrode with an integrated pull-tab for structurally securing an input wire to the electrode and for use in easily removal of the electrode.

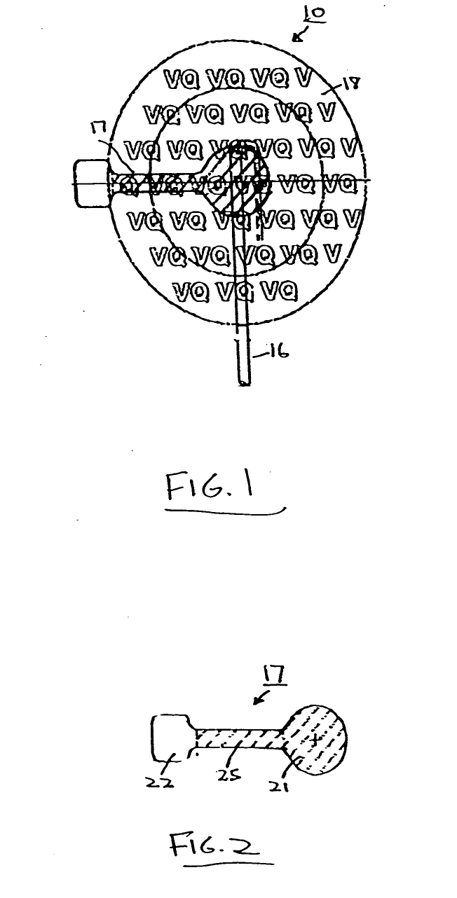

FIG. 1 depicts an electrode according to one embodiment of the present invention. As seen in FIG. 1, a top view of electrode 10 is shown. In this view, laminate layer 18 is provided on top and is shown to be transparent in this embodiment for explanation purposes. Underneath laminate layer 18 is integrated pull-tab layer 17 which secures input wire 16 to electrode 10, and which provides a grip portion outside of the boundary of laminate layer 18 for a user to grab when applying and/or removing the electrode. Although not shown in FIG. 1, electrode 10 also includes a conductive layer that is positioned below laminate layer 18, pull-tab layer 17, and input wire 16. Each of the foregoing components of electrode 10 will be discussed in more detail below.

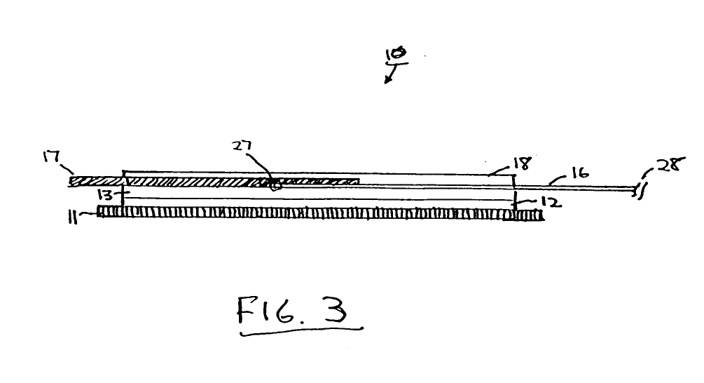

FIG. 2 provides a more detailed top view of pull-tab layer 17. As seen in FIG. 2, pull-tab layer 17 includes disc portion 21 at one end, middle portion 25, and grip portion 22 at the end opposite of disc portion 21. Pull-tab layer 17 is preferably made from a material such as mylar, plastic, polyester, or some other suitable material. As shown by the cross-hatching in FIG. 2, pull-tab layer 17 has an adhesive surface covering the underneath of pull-tab layer 17 so that pull-tab layer 17 securely adheres to the conductive layer of electrode 10, and also securely fastens a connection end of input wire 16 to the conductive layer of electrode 10.

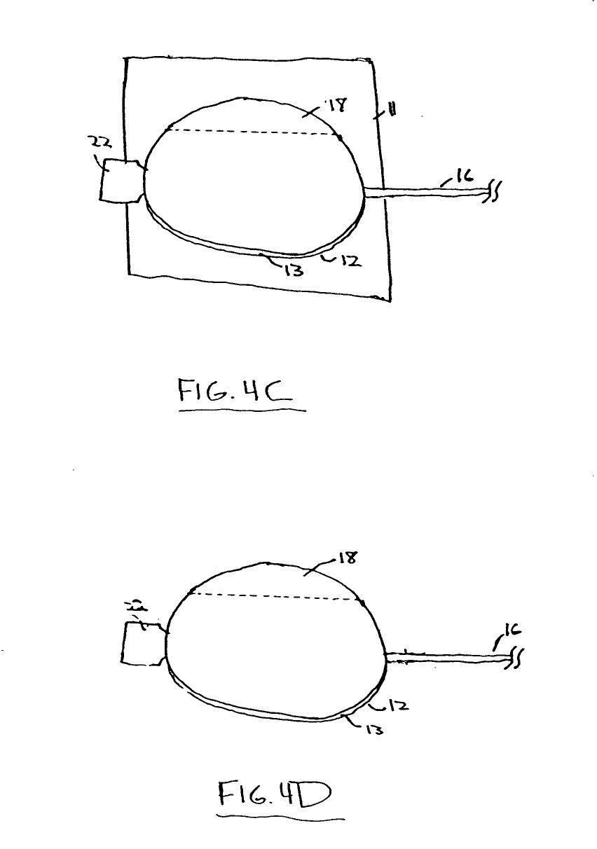

The adhesive surface of pull-tab layer 17 is preferably a known type of glue material for use in a health care environment and has a tacky characteristic, such as a self-adhesive bandage. Prior to being positioned into electrode 10, pull-tab layer 17 preferably has a liner layer placed over the adhesive surface on middle portion 25 and disc portion 21 to maintain the adhesive surface in a good condition before being applied to input wire 16 and the conductive layer. In this regard, the liner layer is pulled from pull-tab layer 17 so that it can be integrated into electrode 10 during production along with input wire 16. This method of integrating pull-tab layer 17 into electrode 10 is discussed in more detail below with respect to FIGS. 4A to 4D.

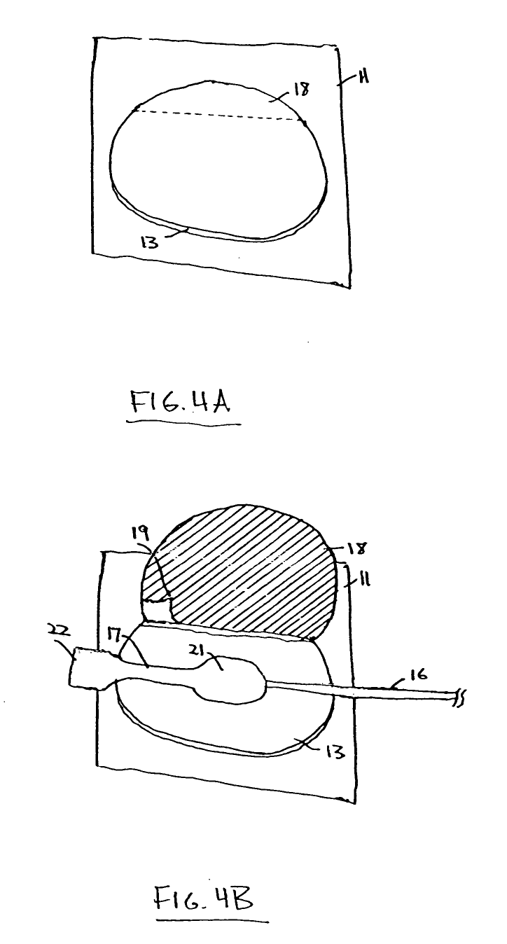

FIG. 3 provides a side view of electrode 10 showing pull-tab layer 17 and input wire 16 integrated between the conductive layer and the laminate layer. As seen in FIG. 3, electrode 10 is comprised of several layers of materials. Starting at the bottom layer in FIG. 3, there is shown removable liner layer 11 which is preferably made of a mylar, polyester, plastic or other suitable material. Removable liner layer 11 is removed from electrode 10 before electrode 10 is applied to the skin of the user. Just above removable liner layer 11 are adhesive surface 12 and conductive layer 13. Adhesive surface 12 is provided on the bottom of conductive layer 13 for attaching electrode 10 to the skin of the user. Preferably, adhesive surface 12 is comprised of a hydrogel material, or some other known conductive material used in the health care field for removably adhering electrodes to a user's skin. It is important that adhesive surface 12 have sufficient adhesive force to securely attach electrode 10 to the user's skin so that the stimulation input signal from input wire 16 is transmitted through conductive layer 13 and adhesive surface 12 to the user's skin.

Conductive layer 13 is preferably made of a conductive carbon material, and the top surface of conductive layer 13, which is opposite adhesive surface 12, is covered with a conductive material, which preferably includes a silver-based material, such as a light silver-based film. As mentioned above, conductive layer 13 is used to adhere electrode 10 to the user's skin and to pass the stimulation input signal from input wire 16 through adhesive surface 12 to the user's skin.

As further seen in FIG. 3, input wire 16 is secured to the conductive material on conductive layer 13 by use of pull-tab layer 17. In particular, connection end 27 of input wire 16 is secured to the center of conductive layer 13 by disc portion 21 of pull-tab layer 17. In this regard, middle portion 25 and disc portion 21 of pull-tab layer 17 are secured to conductive layer 13 by the adhesive surface provided on the bottom of pull-tab layer 17. Grip portion 22 of pull-tab layer 17 extends out beyond the perimeter of conductive layer 13 for easy access by the user of electrode 10 to apply and remove electrode 10 from removable liner layer 11 or from the user's skin. External end 28 of input wire 16 is positioned beyond the perimeter of conductive layer 13 for connection to an electrical power source (not shown) which provides the stimulation signal to input wire 16.

At the very top of electrode 10 shown in FIG. 3 is laminate layer 18 which is used to further secure pull-tab layer 17 and input wire 16 to conductive layer 13, and to provide overall structural integrity to electrode 10. Laminate layer 18 has an adhesive material disposed on the underside surface which faces conductive layer 13 to adhere laminate layer 18 to conductive layer 13, with pull-tab layer 17 and input wire 16 sandwiched between laminate layer 18 and conductive layer 13. The adhesive material provided on laminate layer 18 is preferably a known type of glue material for use in a health care environment and has a tacky characteristic, such as that of a self-adhesive bandage. A liner layer is preferably placed over the adhesive material on laminate layer 18 to maintain the adhesive material in a good condition before laminate layer 18 is positioned over and applied to conductive layer 13. The liner layer is removed from the adhesive material on laminate layer 18 just before laminate layer 18 is positioned over and applied to conductive layer 13.

Accordingly, electrode 10 is comprised of multiple layers for structural integrity, for securely attaching to the skin of a user, and for efficiently conducting the stimulation signal from input wire 16 to the skin of the user. In this manner, pull-tab layer 17 is integrated into electrode 10 for structurally securing input wire 16 to electrode 10 and for use in easy application and removal of electrode 10.

FIGS. 4A through 4D depict a method in which electrode 10 is constructed according to one embodiment of the invention. As seen in FIG. 4A, electrode 10 is shown during production before pull-tab layer 17 and input wire 16 are integrated therein. Removable liner layer 11 is shown to be square in FIGS. 4A to 4D, although any shape can be used so long as the area can accommodate the footprint area of conductive layer 13 of electrode 10. Turning back to FIG. 4A, conductive layer 13 is shown to be attached to removable liner layer 13 by adhesive surface 12 (not shown). Disposed on top of conductive layer 13 is laminate layer 18, although the adhesive material on the bottom surface of laminate layer 18 is covered by a liner at this stage of production. Accordingly, laminate layer 18 is not yet secured to conductive layer 13. Preferably, a large portion of the adhesive material on laminate layer 18 is covered by a removable liner, and the smaller remaining portion is not covered by a liner but is adhered directly to conductive layer 13. The split between these portions of the adhesive material on laminate layer 18 is shown by the dotted line in FIG. 4A.

Next, in FIG. 4B, the portion of laminate layer 18 that is not yet adhered to conductive layer 13 is lifted up, and the removable liner on laminate layer 18 is peeled off (shown at corner 19) to expose the adhesive material on the underside of laminate layer 18. While laminate layer 18 is still lifted over conductive layer 13, connection end 27 is placed over the center of conductive layer 13, and disc portion 21 of pull-tab layer 17 is positioned directly over connection end 27. At this point, any liner layer provided on pull-tab layer 17 has been removed so that middle portion 25 and disc portion 21 adhere to the conductive material of conductive layer 13.

Turning to FIG. 4C, once pull-tab layer 17 and input wire 16 are positioned in place on conductive layer 13, laminate layer 18 is adhered to conductive layer 13, thereby sandwiching disc portion 21 and middle portion 25 of pull-tab layer 17 between laminate layer 18 and conductive layer 13, along with connection end 27 of input wire 16. In this manner, disc portion 21 securely attaches connection end 27 to conductive layer 13, and laminate layer 18 further secures disc portion 21 to conductive layer 13. As seen in FIG. 4C, grip portion 22 of pull-tab layer 17 extends out beyond the border perimeter of laminate layer 18 and conductive layer 13, both of which are of similar shape and size. Grip portion 22 is therefore readily accessible to the user to grab onto when applying and/or removing electrode 10 from removable liner layer 11 or from the user's skin.

FIG. 4D shows electrode 10 when it is ready for application to the user's skin. As seen in FIG. 4D, the user has gripped grip portion 22 to remove electrode 10 from removable liner layer 11 and to apply adhesive surface 12 of conductive layer 13 directly to the user's skin. When stimulation therapy is complete, the user again grips grip portion 22 to remove electrode 10 from the skin and to place electrode 10 back onto removable liner layer 11. Accordingly, adhesive surface 12 of conductive layer 13 is preserved in good condition for the next use of electrode 10, and input wire 16 remains secured to conductive layer 13 in electrode 10 throughout the application and removal process.

In this manner, the present invention provides a multi-layer electrode having an integrated pull-tab which has a round disk at one end to securely connect the wire to the center of the electrode with an increased resistance to pull force and to maintain the connection end of the wire in good electrical contact at the center of the electrode. The pull-tab of the present invention also provides a grip tab at the other end and outside the electrode perimeter for easy gripping by the user for application and/or removal of the electrode from the storage liner or the skin of the user.

The foregoing description of the of the invention has been presented for the purposes of illustration and description, and is not intended to be exhaustive or to limit the scope of the invention to the precise embodiments disclosed herein. It should be appreciated that many modifications, variations and other embodiments of the invention are possible in view of the foregoing description, without departing from the scope or spirit of the invention.

What is claimed:

1. An electrode for delivering a stimulation signal to a user, the electrode comprising:
a conductive layer having an adhesive surface on a first side and a conductive material on a second side;
a laminate layer having an adhesive material on a first side and being disposed over the second side of the conductive layer such that the first side of the laminate layer adheres to the second side of the conductive layer;
an input wire disposed between the conductive layer and the laminate layer, the input wire having a connection end and an external end, the connection end being disposed on the second side of the conductive layer and the external end being provided beyond the boundaries of the conductive layer and the laminate layer; and
a pull-tab layer disposed between the conductive layer and the laminate layer, a disc portion with an adhesive surface being provided at a first end of the pull-tab layer, and a grip portion being provided at a second end of the pull-tab layer, the adhesive surface of the disc portion being disposed over the connection end of the input wire on the conductive layer, and the grip portion extending beyond the boundaries of the conductive layer and the laminate layer.

2. The electrode of claim 1, further comprising a removable liner layer disposed on the adhesive surface of the conductive layer.

3. The electrode of claim 1, wherein the conductive layer and the laminate layer are substantially similar in size.

4. The electrode of claim 1, wherein the conductive layer and the laminate layer are disc shaped and have matching boundaries.

5. The electrode of claim 1, wherein the connection end of the input wire and the disc portion of the pull-tab layer are disposed over the center of the conductive surface of the conductive layer.

6. The electrode of claim 1, wherein the pull-tab layer has a middle portion disposed between the disc portion and the grip portion, and the middle portion has an adhesive surface.

7. The electrode of claim 6, wherein the adhesive surface of the middle portion and the adhesive surface of the disc portion adhere to the conductive material on a second side of the conductive layer.

8. The electrode of claim 1, wherein the adhesive surface on the conductive layer is comprised of a layer of hydrogel adhesive material.

9. The electrode of claim 1, wherein the conductive layer is a conductive carbon film, and the conductive material includes a silver material film.

10. The electrode of claim 2, wherein the removable liner layer is comprised of a mylar material.

11. The electrode of claim 2, wherein the removable liner layer is comprised of a polyester material.
